SEQUENCE LISTING (13 sequences)

SEQ ID NO: 1 - DNA, 1414 bp, Homo sapiens
gccaggaccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttga

SEQ ID NO: 2 - DNA, 1259 bp, Homo sapiens
gccaggaccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatgtgagtgacctggagagaggggctgggagaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttga

SEQ ID NO: 3 - DNA, 1204 bp, Homo sapiens
aggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatccca

SEQ ID NO: 4 - protein, 404 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 5 - protein, 342 aa, Homo sapiens
MAAGTAVGAWVLVLSLWGAVVGAQNITARIGEPLVLKCKGAPKKPPQRLEWKLGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDVSDLERGAGRTRRGGANCRLCGRIRAGNSSPGPGDPGRPGDSRPAHWGHLVAKAATPRRGEEGPRKPGGRGGACRTESVGGT

SEQ ID NO: 6 - protein, 390 aa, Homo sapiens
MAAGTAVGAWVLVLSLWGAVVGAQNITARIGEPLVLKCKGAPKKPPQRLEWKLGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 7 - DNA, 1436 bp, Homo sapiens
gtccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagcccccacaatgatgattaaacacctgacacatctcaaaaaaaaaaaaaaaaaaaaaaaaaaaaaa

SEQ ID NO: 8 - protein, 404 aa, Homo sapiens; identical to SEQ ID NO: 4

SEQ ID NO: 9 - protein, 318 aa, Artificial Sequence
  Synthetic
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGT

SEQ ID NO: 10 - DNA, 35 bp, Artificial Sequence
  Synthetic
ctgacctatgcggccgctgctcaaaacatcacagc

SEQ ID NO: 11 - DNA, 50 bp, Artificial Sequence
  Synthetic
gactgaattcatcagtgatgatggtgatggtgagttcccagccctgatcc

SEQ ID NO: 12 - DNA, 1038 bp, Artificial Sequence
  Synthetic
atgaaaaagacagctatcgcgattgcagtggcactggctggtttcgctaccgttgcggccgctgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactcaccatcaccatcatcactga

SEQ ID NO: 13 - protein, 345 aa, Artificial Sequence
  Synthetic
MKKTAIAIAVALAGFATVAAAAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTHHHHHH

(19) United States
(12) Patent Application Publication
(10) Pub. No.: US 2010/0034897 A1
(43) Pub. Date: Feb. 11, 2010

(54) Method for Removal or Inactivation of Heparin

(75) Inventors: Guillermo A. Ameer, Chicago, IL (US); Yangrong Zhang, Antioch, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: NORTHWESTERN UNIVERSITY, Evanston, IL (US)

(21) Appl. No.: 12/505,985

(22) Filed: Jul. 20, 2009

Related U.S. Application Data
(60) Provisional application No. 61/082,728, filed on Jul. 22, 2008.

(51) Int. Cl. A61K 35/12; C08B 37/10; A61K 38/17

(52) U.S. Cl. 424/520; 536/123.1; 514/12

(57) ABSTRACT

The present invention relates to the use of immobilized RAGE or portions thereof for removal of heparin and low molecular weight heparin from a fluid sample or from a patient in need of neutralization of anticoagulant activity. The invention provides a method for removal or inactivation of heparin and low molecular weight heparin, as well as a device which utilizes this method.

### CROSS-REFERENCE TO RELATED APPLICATIONS

This application claims the benefit of U.S. Provisional Application No. 61/082,728, filed Jul. 22, 2008, which is incorporated herein by reference in its entirety for all purposes.

### STATEMENT OF GOVERNMENT INTEREST

The invention described herein was made in part with government support under grant number R01 DK063123, awarded by the National Institutes of Health (NIH). The United States Government has certain rights in the invention.

### BACKGROUND OF THE INVENTION

1. Field of the Invention

The present invention relates to the use of immobilized RAGE or portions thereof for removal or inactivation of heparin or low molecular weight heparin from a fluid sample or from a patient in need of neutralization of anticoagulant activity.

2. Background of the Invention

Heparin is one of the most commonly used drugs in clinical medicine. Heparin and its low molecular weight derivatives, commonly referred to as low molecular weight (LMW) heparins, are commonly used as anticoagulants during extracorporeal procedures such as cardiopulmonary bypass, extracorporeal membrane oxygenation, dialysis, plasmapheresis, and hemoperfusion. Heparin and LMW heparins are also used to prevent clotting episodes that can follow a stroke. The anticoagulant activity of heparin must be carefully controlled to prevent potentially fatal bleeding. Over the past 40 years, much effort has been made to find safer methods to counteract this potentially dangerous side effect as well as alternative anticoagulants to heparin. Physicians have traditionally used protamine sulfate to accomplish this, but protamine sulfate can have serious side effects in some patients. These side effects include pulmonary hypertension, systemic hypotension, anaphylactic shock, thrombocytopenia, complement activation, and cytokine release. Protamine sulfate is also dangerous to patients that are allergic to seafood because it is primarily isolated from fish sperm. Furthermore, protamine sulfate is ineffective against LMW heparin. Accordingly, there is need for a method for neutralizing the anticoagulant activity of heparin and its derivatives that is safe to use in patients that are allergic to protamine sulfate and that is effective against LMW heparin.

### SUMMARY OF THE INVENTION

This invention relates to the use of the soluble form of the receptor for advanced glycation end product (sRAGE) as a safe alternative to the use of protamine sulfate. RAGE is a member of the immunoglobulin superfamily of cell surface proteins. sRAGE is the extracellular domain (˜30 kDa) of RAGE. Ligand binding to cell surface RAGE triggers the p21ras/MAP kinase signaling cascade and leads to the activation of the transcription factor NF-κB. This activation, which may be induced by advanced glycation end products (AGEs) or pro-inflammatory RAGE ligands such as s100/calgranulins (Yan S D, Schmidt A M, Anderson G M, et al.: Enhanced cellular oxidant stress by the interaction of advanced glycation end products with their receptors/binding proteins. J Biol Chem 269:9889-9897, 1994; Hofmann M A, Drury S, Fu C, et al.: RAGE mediates a novel proinflammatory axis: a central cell surface receptor for S100/calgranulin polypeptides. Cell 97:889-901, 1999; Hsieh H L, Schafer B W, Weigle B, Heizmann C W: S100 protein translocation in response to extracellular S100 is mediated by receptor for advanced glycation endproducts in human endothelial cells. Biochem Biophys Res Commun 316:949-959, 2004) leads to an increase in the expression of NF-κB controlled genes, including pro-inflammatory cytokines, vasoconstrictors, adhesion molecules (Thomas M C, Forbes J M, Cooper M E: Advanced glycation end products and diabetic nephropathy. Am J Ther 12:562-572, 2005) and osteogenic factors (Wan C, He Q, Li G: Osteoclastogenesis in the nonadherent cell population of human bone marrow is inhibited by rhBMP-2 alone or together with rhVEGF. J Orthop Res 24:29-36, 2006). The multi-pattern binding activity of sRAGE is strongly related to its relatively high isoelectric point as demonstrated by its ability to discriminate among ligands with various degrees of net negative charges. Purification of sRAGE using heparin columns has also been reported (Hanford et al. vol. 279 pp 50019-50024, Journal of Biological Chemistry). Others have reported the antagonistic or inhibitory effect of heparin and LMW heparin on cell surface RAGE-ligand interactions (Myint, et al, Diabetes 55, pp 2510-2522, 2006).

In one embodiment this invention utilizes the fact that LMW heparin has been found to have a mean equilibrium dissociation constant (K_{d}) of 17 nM towards cell surface RAGE. Given the fact that sRAGE is a natural protein in the human body, it is an ideal candidate to neutralize the anticoagulant effects of heparin and LMW heparin, without the side effects associated with protamine sulfate (Kalousova et al Nephrology Dialysis Transplantation vol. 22, pp 2020-2026, 2007).

The invention provides a method for removing heparin from a fluid sample taken from a patient in need of neutralization of heparin anticoagulant activity, the method comprising the steps of:
(a) extracorporeally contacting the fluid sample with a Receptor for Advanced Glycation Endproduct (RAGE) or portion thereof under conditions sufficient to bind heparin, thereby creating a substantially heparin depleted fluid sample, and
(b) returning the heparin depleted fluid sample into the patient.

The invention also provides a method for removing heparin from a fluid sample comprising heparin, the method comprising the step of contacting the fluid sample with a Receptor for Advanced Glycation Endproduct (RAGE) or portion thereof under conditions sufficient to bind heparin, thereby forming a substantially heparin depleted fluid sample.

The invention also provides a method for neutralizing the anticoagulant activity of heparin in a patient, comprising administering a pharmaceutical composition comprising a Receptor for Advanced Glycation Endproduct (RAGE), or portion thereof and a suitable carrier to a patient in an amount sufficient to substantially bind heparin in the patient, thereby substantially removing free heparin from the patient.

The invention further provides a method for the neutralizing anticoagulant activity of heparin, wherein the heparin comprises low molecular weight heparin (LMW heparin).

The invention further provides a method for the neutralizing anticoagulant activity of heparin, wherein the heparin comprises a natural or synthetic polysaccharide of heparin. The polysaccharide of heparin can be, for example, a pentasaccharide.

The invention also provides a method for substantially removing heparin or LMW heparin, wherein substantial removal refers to 75%, 77.5%, 80%, 82.5%, 85%, 87.5, 90%, 92.5%, 95%, 97.5%, or 100% removal of heparin or LMW heparin.

The invention further provides a method for neutralizing the anticoagulant activity of heparin, comprising the use of RAGE or a portion thereof, wherein the RAGE comprises soluble RAGE (sRAGE).

The invention further provides a method for neutralizing the anticoagulant activity of heparin, comprising the use of sRAGE, wherein the sRAGE is immobilized onto a substrate.

The invention further provides a method for neutralizing the anticoagulant activity of heparin, comprising the use of RAGE, wherein the RAGE or a portion thereof is immobilized onto a substrate.

The invention further provides a method for neutralizing the anticoagulant activity of heparin, wherein the heparin has a molecular weight ranging from about 5 kDa to about 40 kDa.

The invention further provides a method for neutralizing the anticoagulant activity of LMW heparin wherein the LMW heparin has a molecular weight ranging from about 1500 Da to about 9000 Da.

The invention further provides a method for neutralizing the anticoagulant activity of heparin, comprising the use of RAGE or a portion thereof, wherein the RAGE or a portion thereof is conjugated to a water soluble macromolecule.

The invention further provides a method for neutralizing the anticoagulant activity of heparin, comprising the use of sRAGE; the sRAGE is conjugated to a water soluble macromolecule

The invention further provides a method for neutralizing the anticoagulant activity of heparin, comprising the use of sRAGE, RAGE or a portion thereof, wherein the sRAGE, RAGE, or portion thereof immobilized onto a substrate, wherein the substrate is selected from the group consisting of a particle, a membrane, and a polymeric compound.

The invention further provides a method for neutralizing the anticoagulant activity of heparin, comprising the use of sRAGE, RAGE, or a portion thereof wherein the sRAGE, RAGE or portion thereof immobilized onto a substrate, wherein the substrate is a polymeric compound, wherein the polymeric compound is agarose.

The invention further provides a method for neutralizing the anticoagulant activity of heparin comprising the use of RAGE, wherein the RAGE is selected from the group consisting of SEQ ID NO: 4, SEQ ID NO: 5, SEQ ID NO: 6, SEQ ID NO: 8 and SEQ ID NO: 9.

The invention further provides a method for neutralizing anticoagulant activity of heparin comprising the use of RAGE, wherein the RAGE is encoded by a polynucleotide selected from the group consisting of SEQ ID NO: 1, SEQ ID NO: 2, SEQ ID NO: 3, SEQ ID NO: 7 and complements thereof.

### BRIEF DESCRIPTION OF THE DRAWINGS

FIG. 1 shows the binding capacity of agarose-immobilized RAGE for heparin and LMW heparin. 25 μl of agarose beads were incubated with 2 units of heparin or LMW heparin. The black bars represent the removal of heparin by agarose-immobilized RAGE and the gray bars show the nonspecific removal by plain agarose beads. Error bars represent standard error of the mean from four different preps. The binding capacity of agarose-immobilized RAGE was 54.9±1.7 unit/ml for heparin and 26.2±0.8 unit/ml for LMW heparin. The nonspecific binding to plain agarose beads was negligible.

FIG. 2 shows the kinetics of heparin removal from saline at a flow rate of 250 ml/min. The black squares and line represent samples taken out at the inlet of the hollow fiber device and the gray triangles and line represent samples taken at the outlet of the hollow fiber device. Error bars represent standard error of the mean from two replicates. The data from the inlet can be fit to a two-phase exponential decay. Within one hour, all heparin is removed from the solution.

FIG. 3 shows the kinetics of heparin removal with plain agarose beads at a flow rate of 250 ml/min. The black squares and line represent samples taken at the inlet of the hollow fiber device and the gray triangles and line represent samples from the outlet of the hollow fiber device. Error bars represent standard error of the mean from two replicates. There is only 20% reduction in the heparin concentration which may be contributed by the system dilution.

FIG. 4 shows the kinetics of LMW heparin removal from saline at a flow rate of 250 ml/min. The black squares and line represent samples taken out at the inlet of the hollow fiber device and the gray triangles and line represent samples taken at the outlet of the hollow fiber device. The same agarose-immobilized RAGE used in FIG. 2 was regenerated and reused here. Error bars represent standard error of the mean from two replicates. The data from the inlet can be fit to a two-phase exponential decay. Within one hour, all LWM heparin is removed from the solution.

FIG. 5 shows the clotting kinetics of re-calcified plasma samples spiked with varying concentrations of heparin but treated with agarose-immobilized RAGE prior to the clotting assay. The black squares represent samples spiked with 0.25 unit/ml heparin, black circles represent samples spiked with 0.5 units/ml heparin, black crosses represent samples spiked with 1 unit/ml heparin and black diamonds represent samples spiked with 2 unit/ml heparin. Gray upper triangles represent plasma samples that contained no heparin. Each data point represents the mean from five replicates.

FIG. 6 shows the clotting kinetics of plasma samples spiked with varying concentrations of heparin without treatment with agarose-immobilized RAGE prior to the assay. The black squares represent samples spiked with 0.25 unit/ml heparin, black circles represent samples spiked with 0.5 units/ml heparin, black crosses for 1 unit/ml heparin and black diamonds for 2 unit/ml heparin. Each data point represents the mean from five replicates. Except for slight clotting for samples spiked with 0.25 unit/ml, there was no clotting for samples that were not treated with the immobilized RAGE.

FIG. 7 shows the clotting kinetics of plasma samples spiked with heparin and soluble RAGE. The black circles represents samples spiked with 0.67 units/ml of heparin and 80 μg/ml soluble RAGE and the gray triangles represent control sample spike with 0.5 units/ml only. Each data point represents the mean from five replicates.

FIG. 8 shows the clotting kinetics of plasma samples spiked with varying molar ratio of heparin to soluble RAGE. The black squares represent samples spiked with heparin and soluble RAGE with molar ratio of 1:10, black circles represent 1:8, black diamonds represent 1:6, black crosses represent 1:4, black stars represent 1:2 and black pluses represent 1:1. The gray lower triangles represent a control sample spiked with 0.5 units/ml only. The gray upper triangles represent a plasma control sample without any heparin. Each data point represents the mean from six replicates.

FIG. 9 shows the clotting kinetics of plasma samples spiked with varying molar ratio of LMW heparin to soluble RAGE. The black squares represent samples spiked with LMW heparin and soluble RAGE with molar ratio of 1:2, the black circles represent a molar ratio of 1:1 and the black diamonds represents a molar ratio of 3:1. The gray crosses represent a control sample spiked with 0.5 units/ml only. The gray triangles represent a plasma control sample without any heparin. Each data point represents the mean from three replicates.

FIG. 10 shows the clotting kinetics of re-calcified plasma samples spiked with various concentrations of LMW heparin. The black squares, circles and diamonds represent samples spiked with heparin and then treated with agarose-immobilized RAGE prior to the clotting assay. The black squares represent samples spiked with 0.5 unit/ml heparin, the black circles represent samples spiked with 1 units/ml and the black diamonds represent samples spiked with 2 unit/ml heparin. The black crosses, stars and pluses represent samples spiked with various concentrations of heparin without treatment with agarose-immobilized RAGE prior to the assay. The black crosses represent samples spiked with 0.5 unit/ml heparin, the black stars represent samples spiked with 1 unit/ml and black pluses represent samples spiked with 2 unit/ml heparin. The gray triangles represent plasma samples that contained no heparin. Each data point represents the mean from six replicates.

FIG. 11 shows the clotting kinetics of plasma samples spiked with varying molar ratios of LMW heparin to soluble RAGE. The black squares represent samples spiked with LMW heparin and soluble RAGE with molar ratio of 1:10, the black circles represent a molar ratio of 1:8, the black diamonds represent a molar ratio of 1:4, the black crosses represents a molar ratio of 1:2, the black stars represent a molar ratio of 1:1 and the black pluses represent a molar ratio of 2:1. The gray lower triangles represent a control sample spiked with 1 unit/ml LMW heparin only. The gray upper triangles represent a plasma control sample without any heparin. Each data point represents the mean from three replicates.

FIG. 12 shows the percentage of active heparin in blood samples spiked with varying molar ratios of heparin (1 unit/ml) to soluble RAGE. The percentage of active heparin was calculated from the whole blood re-calcification times measured by Hemochron™ 801. Error bars represent standard error of the mean from ten replicates.

FIG. 13 shows the percentage of active LMW heparin in plasma samples spiked with varying molar ratios of LMW heparin (1 unit/ml) to soluble RAGE. The concentration of active LMW heparin remaining in the plasma samples was measured by anti-Xa assay. Error bars represent standard error of the mean from eight replicates.

FIG. 14 shows the kinetics of heparin removal from human blood at a flow rate of 250 ml/min. The samples were taken out at the inlet of the hollow fiber device. The concentration of heparin remaining in the blood samples was measured by whole blood re-calcification time assay. Error bars represent standard error of the mean from four replicates. The data can be fit to a two-phase exponential decay. Within one hour, all heparin is removed from the blood.

FIG. 15 shows the kinetics of LMW heparin removal from human blood at a flow rate of 250 ml/min. The samples were taken out at the inlet of the hollow fiber device. The concentration of LMW heparin remaining in the plasma samples was measured by anti-Xa assay. Error bars represent standard error of the mean from four replicates. The data can be fit to a two-phase exponential decay. Within one hour, all LMW heparin is removed from the blood.

### DETAILED DESCRIPTION OF THE INVENTION

Specific embodiments of the present invention will become evident from the following more detailed description of certain embodiments and the claims.

The section headings herein are for organizational purposes only and are not to be construed as limiting the subject matter described therein. All references cited in this application are expressly incorporated by reference herein.

### DEFINITIONS

The term “fluid sample” refers to, but is not limited to, human whole blood, human plasma, sera, ascites, lymph, intra-articular fluids, fractional ingredients derived from these fluids, or any liquid ingredient originating in a human patient in which heparin or low molecular weight heparin (LMW heparin) can be found.

The term “low molecular weight heparin” is defined as heparin consisting of only short chains of polysaccharide. LMW heparins are heparin salts having an average molecular weight of less than 8000 Da and for which at least 60% of all chains have a molecular weight less than 8000 Da.

The term “polysaccharide of heparin” is defined as a short polysaccharide that is derived from heparin either naturally via enzymatic or chemical reaction or synthetically via polymerization and/or modification of saccharides using methods known in the art.

The phrase “neutralization of heparin anticoagulant activity” refers to a process in which heparin or low molecular weight heparin is substantially removed or inactivated from a fluid sample or from a patient, thereby allowing for coagulation in the fluid sample or patient.

The phrase “substantial removal” refers to 75%, 77.5%, 80%, 82.5%, 85%, 87.5, 90%, 92.5%, 95%, 97.5%, or 100% removal of heparin or LMW heparin.

The term “inactivation” refers to heparin binding to soluble RAGE (sRAGE) polypeptide, thereby preventing heparin's binding to human Antithrombin III (ATIII).

The term “fragment” as used herein describes a portion, a region, and/or a domain of a molecule, such as, for example, a polynucleotide molecule or a polypeptide molecule. The fragment can be a portion, a region, and/or a domain of the molecule as disclosed herein. Such domains, regions and portions of a polypeptide and/or protein are well known to those of skill in the art and can include, but are not limited to, extracellular domains, transmembrane domains, intracellular domains, enzyme active catalytic sites, protein-protein interacting domains, protein-phospholipid interacting domains, polynucleotide-binding domains and the like. Fragments can, for example, duplicate only a part of the continuous amino acid sequence or secondary conformations within RAGE or sRAGE, or can be the V-domain of RAGE. Preferably the fragment has RAGE activity and has heparin-binding, low molecular weight heparin-binding, or polysaccharide or heparin-binding activity. Additionally the fragment can be bound to a substrate. As used herein, the term “RAGE” encompasses all fragments of RAGE polypeptide that have RAGE-like activity.

The term “ligand” as used herein can be used to describe any molecule and/or compound that binds to a receptor and/or bioadsorbent of the invention. The ligand can be naturally-occurring, it can be a native ligand of the receptor, it can be a synthetic ligand of the receptor, or the like.

In a one embodiment, the method uses a recombinant mammalian receptor in a system wherein the receptor binds a ligand in a fluid sample under appropriate and defined binding conditions, thereby depleting the ligand from the sample. The recombinant mammalian receptor can be a fragment of a polypeptide, wherein the polypeptide is selected from the group consisting of SEQ ID NOs: 4, 5, 6, 8, and 9, and wherein the fragment has RAGE activity.

In an additional embodiment, the method comprises variants of the polypeptides and fragments thereof can be used to neutralize heparin anticoagulant activity. Such variants can incorporate alternative amino acid sequences in the polypeptide that do not result in loss of RAGE activity and/or heparin-binding activity. Substitution of amino acids in a polypeptide sequence, either by replacing codons or replacing amino acid residues during peptide synthesis, are well known to those of skill in the art. Such variants are desirable since the encoded polypeptide can have a different binding affinity for heparin that a naturally occurring or native polypeptide. The binding affinity may be less than that or more than that of the naturally-occurring native peptide. Methods for determining binding affinity are well known to those of skill in the art.

Relatedness of Nucleic Acid Molecules and/or Polypeptides

The term “identity,” as known in the art, refers to a relationship between the sequences of two or more polypeptide molecules or two or more nucleic acid molecules, as determined by comparing the sequences. In the art, “identity” also means the degree of sequence relatedness between polypeptide or nucleic acid molecule sequences, as the case may be, as determined by the match between strings of nucleotide or amino acid sequences. “Identity” measures the percent of identical matches between two or more sequences with gap alignments addressed by a particular mathematical model of computer programs (i.e., “algorithms”).

The term “similarity” is a related concept, but in contrast to “identity,” refers to a measure of similarity which includes both identical matches and conservative substitution matches. Since conservative substitutions apply to polypeptides and not nucleic acid molecules, similarity only deals with polypeptide sequence comparisons. If two polypeptide sequences have, for example, 10 out of 20 identical amino acids, and the remainder are all non-conservative substitutions, then the percent identity and similarity would both be 50%. If in the same example, there are 5 more positions where there are conservative substitutions, then the percent identity remains 50%, but the percent similarity would be 75% (15 out of 20). Therefore, in cases where there are conservative substitutions, the degree of similarity between two polypeptide sequences will be higher than the percent identity between those two sequences.

The term “conservative amino acid substitution” refers to a substitution of a native amino acid residue with a normative residue such that there is little or no effect on the polarity or charge of the amino acid residue at that position. For example, a conservative substitution results from the replacement of a non-polar residue in a polypeptide with any other non-polar residue. Furthermore, any native residue in the polypeptide may also be substituted with alanine, as has been previously described for “alanine scanning mutagenesis” (Cunnigham et al., Science 244:1081-85 (1989)). General rules for conservative amino acid substitutions are set forth in Table I.

TABLE I
Conservative Amino Acid Substitutions

| Original Residues | Exemplary Substitutions | Preferred Substitutions |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, Gln, Asn | Arg |
| Met | Leu, Phe, Ile | Leu |
| Phe | Leu, Val, Ile, Ala, | Leu |
| | Tyr | |
| Pro | Ala | Ala |
| Ser | Thr | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

Conservative amino acid substitutions also encompass non-naturally occurring amino acid residues that are typically incorporated by chemical peptide synthesis rather than by synthesis in biological systems. These include peptidomimetics, and other reversed or inverted forms of amino acid moieties.

Conservative modifications to the amino acid sequence (and the corresponding modifications to the encoding nucleotides) are expected to produce RAGE polypeptide having functional and chemical characteristics similar to those of naturally occurring RAGE polypeptide. In contrast, substantial modifications in the functional and/or chemical characteristics of RAGE polypeptide may be accomplished by selecting substitutions that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain. Naturally occurring residues may be divided into groups based on common side chain properties:

hydrophobic: norleucine, Met, Ala, Val, Leu, Ile;

neutral hydrophilic: Cys, Ser, Thr;

acidic: Asp, Glu;

basic: Asn, Gln, His, Lys, Arg;

residues that influence chain orientation: Gly, Pro; and

aromatic: Trp, Tyr, Phe.

Non-conservative substitutions may involve the exchange of a member of one of these classes for a member from another class. Such substituted residues may be introduced into regions of the human RAGE molecule that are homologous with non-human RAGE polypeptide, or into the non-homologous regions of the molecule.

Identity and similarity of related nucleic acid molecules and polypeptides can be readily calculated by known methods, including but not limited to those described in Computational Molecular Biology (A. M. Lesk, ed., Oxford University Press 1988); Biocomputing: Informatics and Genome Projects (D. W. Smith, ed., Academic Press 1993); Computer Analysis of Sequence Data (Part 1, A. M. Griffin and H. G. Griffin, eds., Humana Press 1994); G. von Heinle, Sequence Analysis in Molecular Biology (Academic Press 1987); Sequence Analysis Primer (M. Gribskov and J. Devereux, eds., M. Stockton Press 1991); and Carillo et al., SIAM J. Applied Math. 48:1073 (1988).

Preferred methods to determine identity and/or similarity are designed to give the largest match between the sequences tested. Methods to determine identity and similarity are codified in publicly available computer programs. Preferred computer program methods to determine identity and similarity between two sequences include, but are not limited to, the GCG program package, including GAP (Devereux et al., Nuc. Acids Res. 12:387 (1984); Genetics Computer Group, University of Wisconsin, Madison, Wis.), BLASTP, BLASTN, and FASTA (Atschul et al., J. Mol. Biol. 215:403-10 (1990)). The BLAST X program is publicly available from the National Center for Biotechnology Information (NCBI) and other sources (Altschul et al., BLAST Manual (NCB NLM NIH, Bethesda, Md.); Altschul et al., 1990, supra). The well-known Smith Waterman algorithm may also be used to determine identity.

By way of example, using the computer algorithm GAP (Genetics Computer Group), two polypeptides for which the percent sequence identity is to be determined are aligned for optimal matching of their respective amino acids (the “matched span,” as determined by the algorithm). A gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 0.1× the gap opening penalty), as well as a comparison matrix such as PAM 250 or BLOSUM 62 are used in conjunction with the algorithm. A standard comparison matrix (see Dayhoff et al., 5 Atlas of Protein Sequence and Structure (Supp. 3 1978) for the PAM250 comparison matrix; see Henikoff et al., Proc. Natl. Acad. Sci USA 89:10915-19 (1992) for the BLOSUM 62 comparison matrix) is also used by the algorithm.

Preferred parameters for polypeptide sequence comparison include the following:
Algorithm: Needleman and Wunsch, J. Mol. Biol. 48:443-53 (1970)
Comparison matrix: BLOSUM 62 from Henikoff et al., Proc. Natl. Acad. Sci. U.S.A. 89:10915-19 (1992)
Gap Penalty: 12
Gap Length Penalty: 4
Threshold of Similarity: 0

The GAP program is useful with the above parameters. The aforementioned parameters are the default parameters for polypeptide comparisons (along with no penalty for end gaps) using the GAP algorithm.

Preferred parameters for nucleic acid molecule sequence comparison include the following:
Algorithm: Needleman et al., J. Mol Biol. 48:443-53 (1970)
Comparison matrix: matches=+10, mismatch=0
Gap Penalty: 50
Gap Length Penalty: 3

The GAP program is also useful with the above parameters. The aforementioned parameters are the default parameters for nucleic acid molecule comparisons.

Other exemplary algorithms, gap opening penalties, gap extension penalties, comparison matrices, thresholds of similarity, etc. may be used by those of skill in the art, including those set forth in the Program Manual, Wisconsin Package, Version 9, September, 1997. The particular choices to be made will depend on the specific comparison to be made, such as DNA to DNA, protein to protein, protein to DNA; and additionally, whether the comparison is between given pairs of sequences (in which case GAP or BestFit are generally preferred) or between one sequence and a large database of sequences (in which case FASTA or BLASTA are preferred).

In addition to generating silent or conservative substitutions as noted, above, the present invention optionally includes methods of modifying the sequences of the Sequence Listing. In the methods, nucleic acid or protein modification methods are used to alter the given sequences to produce new sequences and/or to chemically or enzymatically modify given sequences to change the properties of the nucleic acids or proteins.

Thus, in one embodiment, given nucleic acid sequences are modified, for example, according to standard mutagenesis or artificial evolution methods to produce modified sequences. The modified sequences may be created using purified natural polynucleotides isolated from any organism or may be synthesized from purified compositions and chemicals using chemical means well know to those of skill in the art. For example, Ausubel, (Current Protocols in Molecular Biology, Ausubel et al., eds., Green Publishers Inc. and Wiley and Sons, 1994) provides additional details on mutagenesis methods. Artificial forced evolution methods are described, for example, by Stemmer (1994) Nature 370:389-391, Stemmer (1994) Proc. Natl. Acad. Sci. 91: 10747-1075 I, and U.S. Pat. Nos. 5,811,238, 5,837,500, and 6,242,568. Methods for engineering synthetic transcription factors and other polypeptides are described, for example, by Zhang et al. (2000) J. Biol. Chem. 275:33850-33860, Liu et al. (2001) J. Biol. Chem. 276:11323-11334, and Isalan et al. (2001) Nature Biotechnol. 19: 656-660. Many other mutation and evolution methods are also available and expected to be within the skill of the practitioner.

Similarly, chemical or enzymatic alteration of expressed nucleic acids and polypeptides can be performed by standard methods. For example, sequence can be modified by addition of lipids, sugars, peptides, organic or inorganic compounds, by the inclusion of modified nucleotides or amino acids, or the like. For example, protein modification techniques are illustrated in Ausubel, supra. Further details on chemical and enzymatic modifications can be found herein. These modification methods can be used to modify any given sequence, or to modify any sequence produced by the various mutation and artificial evolution modification methods noted herein.

Accordingly, the invention provides for modification of any given nucleic acid by mutation, evolution, chemical or enzymatic modification, or other available methods, as well as for the products produced by practicing such methods, e.g., using the sequences herein as a starting substrate for the various modification approaches. For example, optimized coding sequence containing codons preferred by a particular prokaryotic or eukaryotic host can be used e.g., to increase the rate of translation or to produce recombinant RNA transcripts having desirable properties, such as a longer half-life, as compared with transcripts produced using a non-optimized sequence. Translation stop codons can also be modified to reflect host preference. For example, preferred stop codons for S. cerevisiae and mammals are TAA and TGA, respectively. The preferred stop codon for monocotyledonous plants is TGA, whereas insects and E. coli prefer to use TAA as the stop codon.

The polynucleotide sequences of the present invention can also be engineered in order to alter a coding sequence for a variety of reasons, including but not limited to, alterations which modify the sequence to facilitate cloning, processing and/or expression of the gene product. For example, alterations are optionally introduced using techniques which are well known in the art, for example, site-directed mutagenesis, to insert new restriction sites, to alter glycosylation patterns, to change codon preference, to introduce splice sites, etc.

Furthermore, a fragment or domain derived from any of the polypeptides of the invention can be combined with domains derived from other transcription factors or synthetic domains to modify the biological activity of a transcription factor. For instance, a DNA-binding domain derived from a transcription factor of the invention can be combined with the activation domain of another transcription factor or with a synthetic activation domain. A transcription activation domain assists in initiating transcription from a DNA-binding site. Examples include the transcription activation region of VP 16 or GAL4 (Moore et al. (1998) Proc. Natl. Acad. Sci. 95: 376-381; and Aoyama et al. (1995) Plant Cell 7: 1773-1785), peptides derived from bacterial sequences (Ma and Ptashne (1987) Cell 51: 113-119) and synthetic peptides (Giniger and Ptashne, (1987) Nature 30: 670-672).

### Expression and Modification of Polypeptides

Typically, polynucleotide sequences of the invention are incorporated into recombinant DNA (or RNA) molecules that direct expression of polypeptides of the invention in appropriate host cells, transgenic plants, in vitro translation systems, or the like. Due to the inherent degeneracy of the genetic code, nucleic acid sequences which encode substantially the same or a functionally equivalent amino acid sequence can be substituted for any listed sequence to provide for cloning and expressing the relevant homologue.

RAGE may be produced using recombinant DNA technology using bacteria, yeast, or mammalian cells. For the purpose of this invention, RAGE can be expressed in E. coli and purified to homogeneity. The polynucleotide encoding RAGE can be, for example, the Homo sapiens advanced glycosylation end product-specific receptor (AGER), transcript variant 1; NM-00 1136.3, GI:26787960 (SEQ ID NO: 1 encoding SEQ ID NO: 4) and/or Homo sapiens advanced glycosylation end product-specific receptor (AGER), transcript variant 2; NM-172 197.1, GI:2678796 1 (SEQ ID NO: 2 encoding SEQ ID NO: 5) and/or Homo sapiens receptor for advanced glycosylation end-products deletion exon 3 variant (AGER) mRNA, complete cds, alternatively spliced; AY755624.1, Gk59799503 (SEQ ID NO: 3 encoding SEQ ID NO: 6) and/or Homo sapiens advanced glycosylation end product-specific receptor cDNA clone; C:22357; IMAGE: 4718076; BC020669.1; GI: 18088362 (SEQ ID NO: 7 encoding SEQ ID NO: 8). The extracellular portion of RAGE is cloned into an E. coli expression vector (pASK40) (Skerra et al. Biotechnology (NY) 9: 273-278 (1991)). Cells are streaked onto LB/Carbenicillin agar plates and grown overnight at 37° C. Colonies are then recovered by washing and the suspension is transferred to 1000 mL of 2×TY media containing 100 μg of carbenicillin. The culture is grown at 37° C. for 4 hours, cooled down to 25° C. for 2 hours before induced with 1 mM IPTG, and grown overnight at 25° C. Cells are harvested and re-suspended in 50 mL of ice cold TES (0.2 M TrisCl, 0.5 mM EDTA, 0.5 M Sucrose, pH 8.0) to which 500 μL of a protease inhibitor cocktail and 500 μL of lysozyme (20 mg/mL in TES) were added. The suspension was then mixed with 100 mL of water and incubated on ice for 60 minutes with gentle shaking and centrifuged to collect the periplasmic fraction. The periplasmic fraction was dialyzed into Ni column buffer A (20 mM TrisCl, 300 mM NaCl, 10 mM Imidazole, pH8.0) and then purified using a HisTrap™ affinity column. The peak fractions were identified on SDS-PAGE gel, filter-sterilized, and dialyzed twice into 2 L of HEPES buffer (10 mM HEPES, 150 mM NaCl, 1 mM EDTA, 0.005% Tween 20, pH 7.4) overnight at 4° C.

Proteins or portions thereof may be produced not only by recombinant methods, but also by using chemical methods well known in the art. Solid phase peptide synthesis may be carried out in a batch-wise or continuous flow process which sequentially adds amino and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-a-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential de-protection, coupling of derivitized amino acids, and washing with dichloromethane and/or N,N-dimethylformamide. The peptide is cleaved between the peptide carboxy terminus and the linker group to yield a peptide acid or amide. (Novabiochem 1997198 Catalog and Peptide Synthesis Handbook, San Diego Calif. pp. S1-20). Automated synthesis may also be carried out on machines such as the ABI 43 1 A peptide synthesizer (PE Biosystems). A protein or portion thereof may be substantially purified by preparative high performance liquid chromatography and its composition confirmed by amino acid analysis or by sequencing (Creighton (1984) Proteins, Structures and Molecular Properties, WH Freeman, New York, N.Y.).

### Immobilization of RAGE

In a useful embodiment of the invention, RAGE is immobilized onto a substrate. RAGE can be for example immobilized through a chemical bond or through a physical bond. RAGE may be bound directly to the substrate or may be bound to the substrate via a linker molecule. Methods for producing RAGE, immobilizing RAGE onto substrates, and systems that comprise immobilized RAGE are described in WO 2007/097922, filed Feb. 7, 2007, which is incorporated by reference in its entirety.

Substrates useful in this embodiment can be comprised of, but are not limited to, a bead, a column, a plastic dish, a plastic plate, a microscope slide, a nylon membrane, a micro-array, a particle, a porous particle, a membrane, a mesh, a dialysis membrane, a multi-well plate or, a polymeric compound. In one embodiment, the substrate is comprised of a synthetic material.

In another embodiment soluble RAGE polypeptide has a domain having binding affinity for an adsorbent. The adsorbent can be a compound having specific binding activity, such as an immunoglobulin or the like, or having non-specific binding activity, such as dextran sulphate, a protein having at least one PDZ domain, or the like.

In another useful embodiment RAGE is immobilized onto agarose gel beads (Sepharose™ CL-4B GE Healthcare, Sunnyvale, Calif.) using the cyanogen bromide (CNBr) surface activation chemistry. The average diameter of the beads is 61.9±15.6 μm. The agarose beads are washed with 10 volumes of ultrapurified water and then re-suspended in an equal volume of ultrapurified water and two volumes of 2 M sodium carbonate. After being chilled on ice for 20 minutes, the agarose suspension is activated for 5 to 7 min with 20% of CNBr (each gram is pre-dissolved in 1.5 ml acetonitrile) with vigorously stirring until the suspension was clear of any undissolved CNBr particles. The activated agarose is then washed sequentially with each of the following ice-chilled buffers: 30 volumes of 1 mM HCl, 30 volumes of ultrapurified water, 20 volumes of sodium bicarbonate buffer (0.1 M NaHCO₃, 0.5 M NaCl, pH 8.3) and 20 volumes of HEPES buffer (10 mM HEPES, 150 mM NaCl, 1 mM EDTA, 0.005% Tween 20). Immediately thereafter, the activated agarose gel is combined with purified sRAGE and the immobilization reaction was carried out for 24 hours on a rocker at 4° C. The unbound RAGE is then removed and the agarose-immobilized RAGE is rinsed four times with 4 volumes of HEPES buffer. The reaction is then quenched with 4 volumes of a glysine buffer (0.2 M glysine, 0.5 M NaCl, 0.1 M NaHCO₃, pH 8.3) overnight on a rocker at 4° C. After quenching, the agarose-immobilized RAGE is rinsed four times with 4 volumes of HEPES buffer and then re-suspended in 4 volumes of HEPES buffer.

In a one embodiment the polypeptide is bound to the substrate via a linker molecule, the linker molecule selected from the group consisting of a thiol group, a sulfide group, a phosphate group, a sulfate group, a cyano group, a piperidine group, an Fmoc group, and a Boc group. Methods for forming such linkages are well known to those of skill in the art (see, for example, Glasser, et al., (1987) Proc. Natl. Acad. Sci. 84:4007, 1987 Jacobs, et al., J. Biol. Chem. 262:9808; Floros, et al., (1986) J. Biol. Chem. 26 1:9029; White, et al., (1985) Nature 3 17.36 1; Glasser, et al., (1988) (a) J. Biol. Chem. 263:9; Glasser, et al., (1988)(b) J. Biol. Chem. 263: 10326; and Jobe et al., (1987) Am. Rev. Resp. Dis. 136: 1032). However, polypeptides can be synthesized on a 0.25 mmol scale with an Applied Biosystems model 431, A peptide synthesizer using a FASTMOC strategy (see Fields, C. G et al., (1991) Peptide Res. 4: 95-101). The peptides can be synthesized with pre-derivatized Fmoc-Gly resin (Calbiochem-Nova, La Jolla, Calif.) or PEG-PA resin (Perceptive Biosystems, Old Connecticut Path, Mass.) and can be single coupled for all residues.

### Binding Interaction Between RAGE and Heparin

Heparin concentrations in a sample can be measured using methods well known to those of skill in the art. Heparin concentration can be measured for example through an assay using Azure II dye. In one embodiment, a 0.1 ml test sample of heparin solution is mixed with 0.9 ml of Azure II dye solution (0.01 mg/ml). The mixture is then incubated at room temperature for 1 min and the absorbance at 500 nm is measured. A standard curve is then generated by using known concentrations of heparin. The linear range of the standard curve is from 0 to 4 units/ml of heparin.

RAGE activity is measured using binding assays well known to those of skill in the art. Binding affinity is expressed as an association constant, Kₐ which is defined as the molar concentration of RAGE-heparin complex divided by the molar concentrations of free heparin and RAGE under equilibrium conditions. Various methods such as Scatchard analysis in conjunction with radioimmunoassay techniques may be used to assess the affinity of RAGE for heparin. RAGE preparations with Kₐ ranging from about 10⁶ to 10¹² l/mole are preferred. RAGE activity can also be measured using nitrocellulose filter binding assays, such as describe by Wilton et al (Wilton et al. Protein Expr. Purif. 47: 25-35 (2006)). To determine the binding capacity of agarose-immobilized RAGE for heparin, 25 μl of immobilized RAGE is re-suspended into 1 ml of PBS buffer spiked with varying concentrations of heparin from 1 unit/ml to 10 unit/ml. At least three replicates are repeated for each sample. The samples are then incubated at 37° C. for 1 hour under vigorous shaking. After incubation, the unbound heparin left in the supernatant is determined with Azure II dye assay. The binding capacity is calculated using the following equation: ρ_{S}=Vₜₒₜₐₗ/V_{gel}(C₀−C_{eq}), where ρ_{S} is the binding capacity (units of heparin per ml of settled gel beads), Vₜₒₜₐₗ is the total volume (liquid+gel) of each sample (1 ml), V_{gel} is the settled-volume of the gel (0.025 ml), C₀ is the initial heparin concentration (units/ml), and C_{eq} is the concentration of unbound heparin at equilibrium (units/ml). Heparin binding capacity can be 25-100 units/ml of agarose gel with the gel containing 3-5 mg RAGE/ml of agarose gel.

A plasma re-calcification clotting assay may be used to characterize the ability of agarose-immobilized RAGE to neutralize the anticoagulant activity of Heparin by measuring the clotting kinetics of re-calcified plasma. Human blood is collected and treated with acid-citrate-dextrose (ACD) as an anticoagulant. In the absence of ACD, clotting kinetics are governed by the presence or absence of heparin in the samples. Platelet poor plasma is prepared by spinning whole blood at 1200 rpm for 15 min to obtain the platelet rich plasma and then spinning the platelet rich plasma at 2000 rpm for 15 min. 0.5 ml of the platelet poor plasma is spiked with varying concentrations of heparin from 0.25 units/ml to 2 units/ml, and then incubated with 80 μl of agarose-immobilized RAGE for one hour at 37° C. with vigorous shaking. After incubation, 150 μl of the supernatants from each sample is added into a 48-well tissue culture plate and quickly mixed with 150 μl of pre-warmed 0.025 M CaCl₂. Five replicates for each sample are then measured. As the plasma clots it becomes turbid. The plate is then immediately placed in a plate reader and absorbance at 405 nm is measured every 30 seconds for one hour.

### Neutralization of Heparin Anticoagulant Activity

In a useful embodiment, the invention provides a system comprising a polypeptide having RAGE activity and a substrate, wherein the polypeptide is chemically bound to the substrate. In a one embodiment the RAGE can bind heparin reversibly under controlled conditions, thereby allowing the system of the invention to be regenerated and used multiple times. In further embodiments, the RAGE polypeptide is soluble in an aqueous environment, in a non-aqueous environment, or in a mixed aqueous and non-aqueous environment. The system can further include a device for use in a clinical setting, such as a clinic or hospital, or can be used outside a building, such as when in use in the field. The system may also be used in vivo, whereby the system is implanted within a lumen or chamber of an organ or tissue of an individual having a disease or disorder.

The system of the invention may be used in a method for depleting a soluble heparin ligand from a fluid sample of an individual having a disease or disorder, the method comprising the steps of: i) providing a fluid sample from an individual in need of heparin removal; ii) incubating the sample with a system comprising a receptor as disclosed herein under appropriate binding conditions; iii) allowing the heparin to bind the receptor and deplete the sample of heparin; and iv) returning the heparin-depleted sample to the individual, thereby depleting the heparin from the fluid sample.

In one embodiment, agarose immobilized RAGE or fragment thereof absorbent and blood or another bodily fluid (e.g., plasma) of a patient is led out from the body is charged into a suitable container (e.g., blood bag) and mixed to thereby remove heparin. The heparin-bound absorbent is removed by conventional filtration methods or other suitable means and the heparin-depleted body fluid is then returned to the patient.

In a further embodiment, immobilized RAGE absorbent can be charged into a device such as one or more suitable columns. Alternatively, RAGE can be immobilized onto one or more columns or membranes. The column or membrane containing RAGE can then be assembled into an extracorporeal circulation system to remove heparin in an online manner. In this case, whole blood or plasma separated from the blood is allowed to pass through the column or membrane to remove heparin. In one aspect, the extracorporeal circulation system may comprise at least one reservoir, at least one inlet tube, at least one outlet tube, and/or at least one pump, RAGE or portions thereof immobilized on a substrate within the reactor, means for retaining the substrate within the reactor, means for re-circulating and agitating or dispersing the re-circulating solution-substrate within the reactor chamber to prevent packing of the substrate, wherein the agitation is limited to avoid subjecting the solution to excessive or damaging forces. While in use, the system is reversibly connected or attached to a fluid line that is in fluid communication with the blood or circulatory system of an individual having a disease or disorder. The pump circulates the blood through the system under conditions that enhance the binding of heparin to the RAGE polypeptide, thereby removing substantial amounts of heparin from the blood. The blood is returned to the individual thereby improving the individual's prognosis. The system also can be used in a manner and at time intervals similar to that used with dialysis devices well known to those in the art.

The Examples that follow are illustrative of specific embodiments of the invention, and various uses thereof. They are set forth for explanatory purposes only, and are not to be taken as limiting the invention.

### Example 1

### Rage Immobilization onto Agarose Gel Beads

The RAGE-His construct (SEQ ID NO: 12, encoding SEQ ID NO: 13) was a generous gift from Dr. Rosemarie Wilton at Argonne National Laboratory, Naperville, Ill. The RAGE-His construct was synthesized as disclosed in Wilton R., et al. (Wilton R., et al. (2006) Protein Expression Purification 47: 25-35; herein incorporated by reference in its entirety). A polynucleotide encoding the extracellular portion of RAGE was cloned into the E. coli expression vector pASK40 having polylinkers as modified by Yuri Londer (Argonne National Laboratory, Argonne, Ill.). The I.M.A.G.E. cDNA clone (clone ID 4718076) containing the complete coding sequence for human RAGE (SEQ ID NO: 7, encoding SEQ ID NO: 8) was obtained from the American Type Culture Collection (ATCC, Manassas, Va.). The polynucleotide encoding the extracellular region of RAGE from amino acid residues 23 through 340 (SEQ TD NO: 9) was amplified using polymerase chain reaction (PCR) with the following oligonucleotides (MWG Biotech, High Point, S.C.): 5′CTGACCTATGCGGCCGCTGCTCAAAACATCACAGC-3′ (SEQ ID NO: 10) and 5′GACTGAATTCATCAGTGATGATGGTGATGGTGAGTTCCCAGCCCTGATCC-3′ (SEQ ID NO: 11). The resulting polynucleotide therefore incorporated a NotI restriction site (single underline in SEQ ID NO: 10) in the region equivalent to the N-terminal portion of the encoded polypeptide sequence and a six-histidine tag followed by two stop codons and an EcoRI restriction site (single underline in SEQ ID NO: 11) in the region equivalent to the C-terminal portion of the encoded polypeptide. PCR was performed using Pfu DNA polymerase (Stratagene, La Jolla, Calif.) following the manufacturer's protocol. The resulting 1004 bp fragment was digested with NotI and EcoRI (Promega, Madison, Wis.). The fragment was ligated in frame with the OmpA signal sequence of pASK40 containing the modified polylinker; T4 ligase was obtained from GibcoBRLAnvitrogen (Carlsbad, Calif.). The recombinant clones were sequenced (performed by MWG Biotech, High Point, S.C.) to confirm identity of the RAGE extracellular domain insert. Plasmids were transformed into E. coli strain JM83 for expression; bacterial stocks were maintained at −80° C. in LB medium containing 100 μg/ml carbenicillin and 15% glycerol.

Cells were then streaked from a frozen glycerol stock of the RAGE-His construct onto LB/carbenicillin agar plates and grown overnight at 37° C. The next morning, colonies were recovered from the plate by washing with 2-3 ml of 2×TY media using a sterile cell scraper to loosen the colonies. The suspension was transferred to 1000 ml of 2×TY media containing 100 pg of carbenicillin. The culture was grown at 37° C., 250 rpm in an orbital shaker for 4 hours and cooled down to 25° C. for 2 hours. The culture was grown at 30° C., 250 rpm in an orbital shaker until the ODaoo was between 0.8-1.0. The culture was induced with 1 mM IPTG (isopropyl-beta-D-thiogalactopyranoside) and grown overnight (approximately 16-18 hours). The cells were harvested by centrifugation (8,000 rpm for 20 minutes) and stored at −80° C. Immediately, prior to purification, the cells were resuspended in 50 ml of icecold TES (0.2 M Tris-HCl, 0.5 mM EDTA, 0.5 M Sucrose, pH 8.0) per liter of culture, to which 500 μL of a protease inhibitor cocktail and 500 μL of lysozyme (20 mg/mL in TES) were added. The suspension was then mixed with 100 mL of water and incubated on ice for 60 minutes with gentle shaking and centrifuged to collect the periplasmic fraction. The periplasmic fraction was dialyzed overnight against Buffer A (20 mM TrisCI, 300 mM NaCl, 10 mM Imidazole). The dialyzed periplasmic fraction was purified using a 5 ml bed volume HISTRAP HP affinity column, (Amersham Biosciences). The column was equilibrated with 10 volumes of Buffer A (20 mM TrisCI, 300 mM NaCl, 10 mM Imidazole). The periplasmic fraction was loaded onto the resin at a flow rate of 2.0 ml/min and run at a gradient of 0-60% Buffer B (20 mM TrisCl, 300 mM NaCI, 500 mM Imidazole). The peak fractions were identified on SDS-PAGE gel. The identified RAGE fractions were pooled; filter sterilized and dialyzed into 2,000 ml of HEPES buffer (10 mM HEPES, 150 mM NaCI, 3 mM EDTA, 0.005% Tween 20) overnight at 4° C. The dialysis buffer was changed the following day and the peak fractions (RAGE) were dialyzed again overnight at 4° C. in 2,000 ml of HEPES buffer.

Ten milliliters of an sRAGE solution (1 mg/ml buffer) was then immobilized onto 2 mL agarose gel beads (Sepharose™ CL-4B GE Healthcare, Sunnyvale, Calif.) using the cyanogen bromide (CNBr) surface activation chemistry. Approximately 5 mg RAGE was loaded per mL beads. The average diameter of the beads that were used is 61.9±15.6 μm. The agarose beads were washed with 10 volumes of ultrapurified water and then re-suspended in an equal volume of ultrapurified water and two volumes of 2 M sodium carbonate. After being chilled on ice for 20 minutes, the agarose suspension was activated for 5 to 7 min with 20% of CNBr (each gram is pre-dissolved in 1.5 ml acetonitrile) with vigorously stirring until the suspension was clear of any undissolved CNBr particles. The activated agarose was then washed sequentially with each of the following ice-chilled buffers: 30 volumes of 1 mM HCl, 30 volumes of ultrapurified water, 20 volumes of sodium bicarbonate buffer (0.1 M NaHCO₃, 0.5 M NaCl, pH 8.3) and 20 volumes of HEPES buffer (10 mM HEPES, 150 mM NaCl, 1 mM EDTA, 0.005% Tween 20). Immediately thereafter, the activated agarose gel is combined with purified sRAGE (typical yield after purification was 10-15 mg/L culture) and the immobilization reaction was carried out for 24 hours on a rocker at 4° C. The unbound RAGE was then removed and the agarose-immobilized RAGE was rinsed four times with 4 volumes of HEPES buffer. The reaction was then quenched with 4 volumes of a glysine buffer (0.2 M glysine, 0.5 M NaCl, 0.1 M NaHCO₃, pH 8.3) overnight on a rocker at 4° C. After quenching, the agarose-immobilized RAGE was rinsed four times with 4 volumes of HEPES buffer and then re-suspended in 4 volumes of HEPES buffer. The immobilization procedure resulted in 2.5-5.0 mg bound RAGE/mL packed agarose gel beads.

### Example 2

### Binding Capacity of Agarose-Immobilized Rage for Heparin

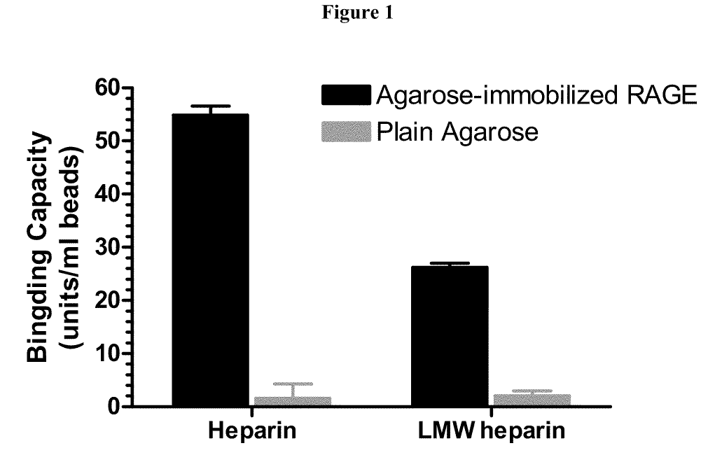

A binding capacity assay was performed to assess the capacity of agarose-immobilized RAGE binding for heparin. 25 μl of immobilized RAGE was mixed with 1 ml of heparin or LMW heparin (Lovenox®) solution at concentrations of 2 unit/ml and the samples were incubated at 37° C. for one hour. The concentration of heparin or LMW heparin was measured by Azure II dye. The results are shown in FIG. 1. The binding capacity was 54.9 unit/ml for heparin and 26.2 unit/ml for LMW heparin. For both of them, nonspecific binding to plain agarose beads was negligible. In this assay, 3 mg of soluble RAGE was immobilized onto each ml of agarose gel beads. The binding capacity was 18 units of heparin per mg of immobilized RAGE. Since each mg of heparin equals to 167 units, the binding capacity can be converted to 0.11 mg of heparin per mg of immobilized RAGE. The molecular weight of heparin is almost half of RAGE. Therefore, the binding molar ratio of heparin to RAGE is 1:5. For LMW heparin, each mg equals to 100 units and the binding molar ratio of LMW heparin to RAGE can be calculated as 2:1. The data suggest the method of using immobilized RAGE to remove heparin and LMW heparin is efficient.

### Example 3

### Neutralization of Heparin Anticoagulant Activity Using Agarose-Immobilized RAGE

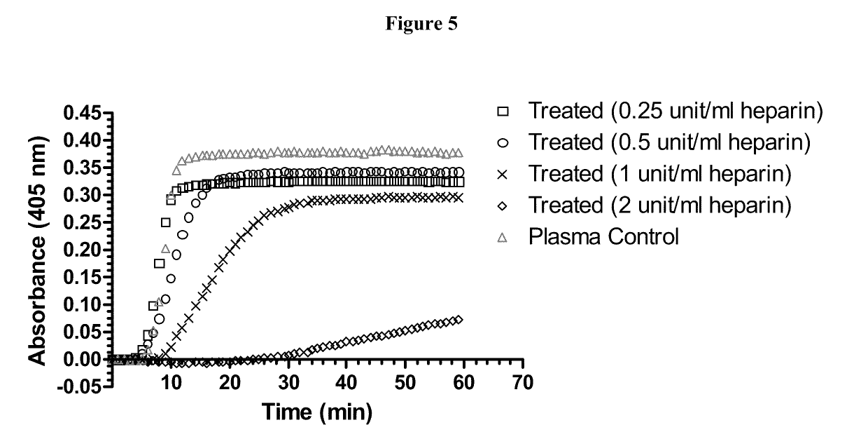

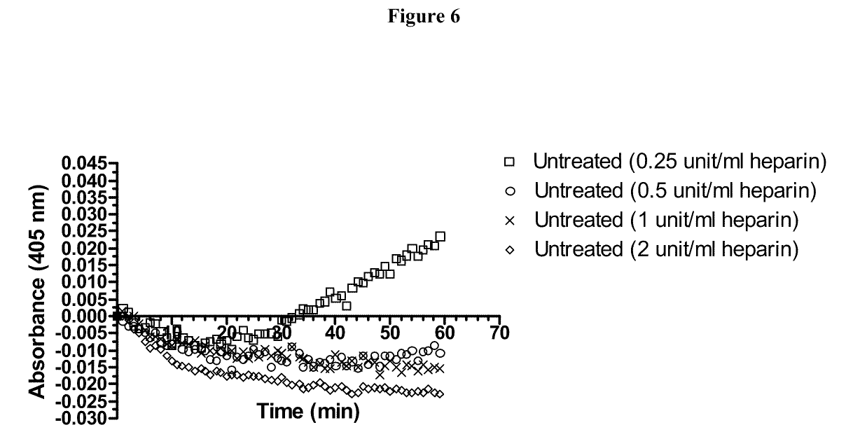

A plasma re-calcification clotting assay was used to demonstrate the ability of immobilized RAGE to neutralize the anticoagulant activity of Heparin. 0.5 ml of plasma was spiked with varying concentrations of heparin: 0.25 unit/ml, 0.5 unit/ml, 1 unit/ml and 2 unit/ml. The samples were then mixed with 80 μl of agarose-immobilized RAGE and incubated at 37° C. for one hour with shaking. Two replicates were included for each sample. Similarly, control plasma samples treated with heparin were prepared (no treatment with agarose-immobilized RAGE) in addition to a plasma control without any heparin. All of the plasma samples were re-calcified to neutralize the ACD anticoagulants by addition of CaCl₂. The absorbance was measured every 30 sec for one hour. The clotting kinetics were plotted as absorbance versus time. FIG. 5 shows the clotting kinetics of recalcified plasma samples that were first spiked with varying concentrations of heparin and then treated with agarose-immobilized RAGE prior to the clotting assay. FIG. 6 shows the kinetics of control samples spiked with heparin but not treated with agarose-immobilized RAGE. All of the samples that were treated with the agarose immobilized RAGE and could completely remove the heparin were able to clot normally. The data confirm that agarose-immobilized RAGE was able to neutralize the anticoagulant activity of heparin.

### Example 4

### Neutralization of Heparin Anticoagulant Activity Using Soluble RAGE

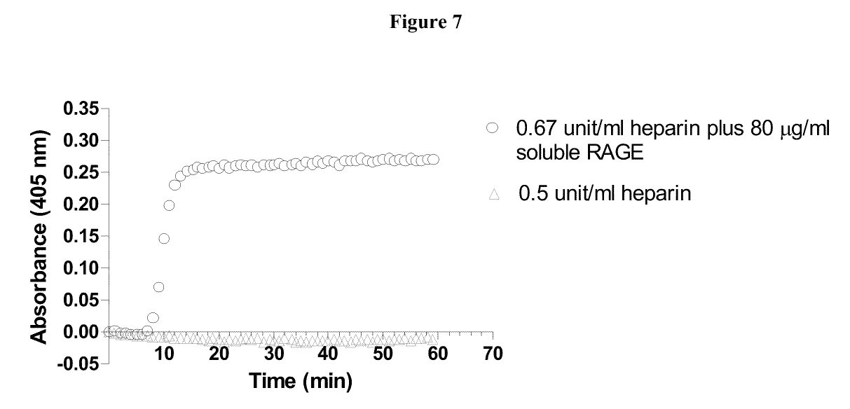

The plasma re-calcification clotting assay was used to demonstrate the ability of soluble RAGE to neutralize the anticoagulant activity of Heparin. The plasma sample was spiked with 0.67 unit/ml of heparin and 80 μg/ml of soluble RAGE, with heparin to RAGE molar ratio of 1:10. The sample was incubated at 37° C. for one hour and then tested in the clotting assay. The data are shown in FIG. 7. The clotting kinetics are similar to those of the control plasma samples that had no heparin (Gray triangles in FIG. 5). Comparing to the heparin control (Gray triangles in FIG. 7), the data confirm that soluble RAGE can neutralize the anticoagulant activity of heparin.

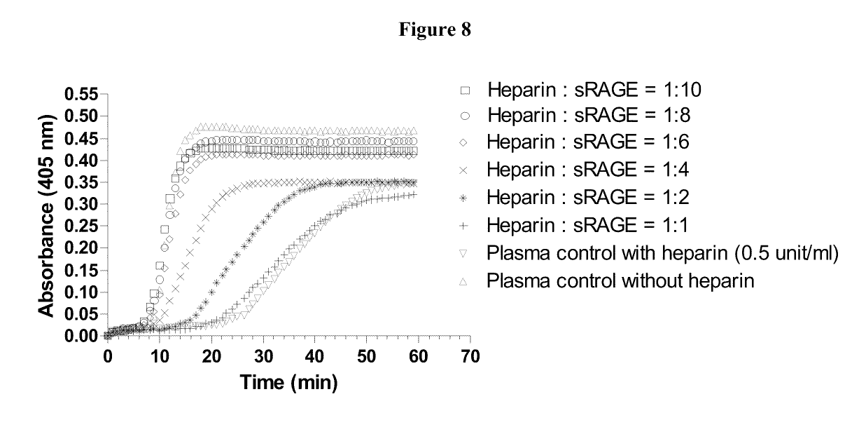

To determine the minimum amount of soluble RAGE required for complete neutralization, plasma samples from another subject were spiked with varying molar ratios of heparin to soluble RAGE. The plasma sample were spiked with 0.5 unit/ml of heparin plus varying concentrations of soluble RAGE so that the molar ratio of heparin to RAGE varied from 1:10 to 1:1. The sample was incubated at 37° C. for one hour and then tested in the clotting assay. As shown in FIG. 8, when the molar ratio of heparin to RAGE was 1:6, the clotting kinetics were similar to the normal plasma control, indicating that this amount of soluble RAGE is sufficient to neutralize all the heparin in the system. In the case of lesser amount of soluble RAGE, the clotting kinetics became slower and slower until they were equal to the heparin control.

### Example 5

### Neutralization of the Anticoagulant Activity of Low Molecular Weight Heparin Using Soluble RAGE

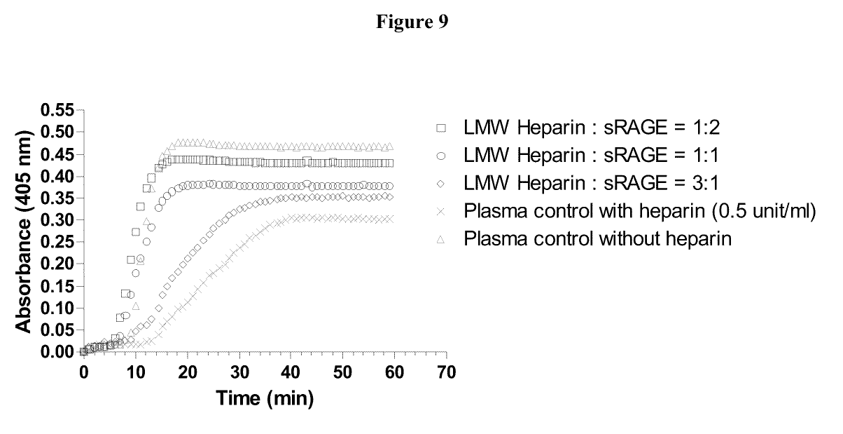

The plasma clotting assay was used to demonstrate the ability of soluble RAGE to neutralize the anticoagulant activity of low molecular weight heparin (LMW heparin). LMW heparin (Sigma Aldrich™) had a molecular weight of 5,665 Da and specific activity of 86 IU/mg. The plasma samples were spiked with 0.5 unit/ml of LMW heparin and varying concentrations of soluble RAGE so that the molar ratios of heparin to RAGE were 1:2, 1:1 and 3:1. The sample was incubated at 37° C. for one hour and then tested in the clotting assay. The data are shown in FIG. 9. When the molar ratios of heparin to RAGE were 1:2 and 1:1, the clotting kinetics were very similar to the normal plasma control. The data confirm that soluble RAGE is able to neutralize the anticoagulant activity of LMW heparin and that it can neutralize equal molar amounts of LMW heparin, which is more efficient than the neutralization of native heparin.

### Example 6

### Neutralization of the Anticoagulant Activity of LMW Heparin Using Agarose-Immobilized RAGE

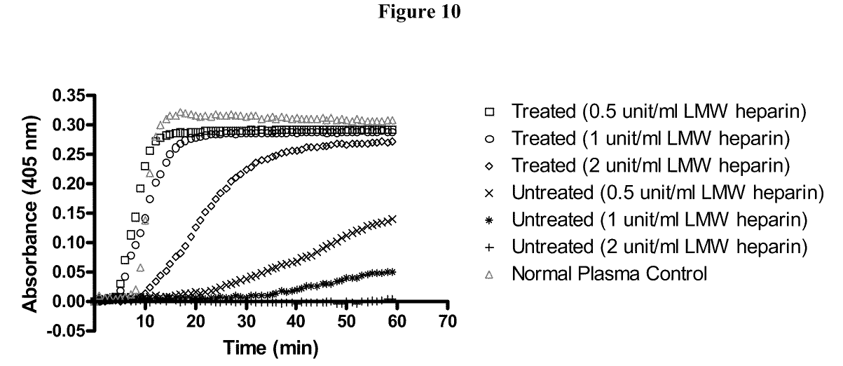

The re-calcified plasma clotting assay was used to confirm the ability of immobilized RAGE to neutralize the anticoagulant activity of LMW heparin. FIG. 10 shows the clotting kinetics of re-calcified plasma samples that were first spiked with various concentrations of LMW heparin and then treated with agarose-immobilized RAGE prior to the clotting assay. The kinetics of control samples spiked with heparin but not treated with agarose-immobilized RAGE are also included in FIG. 10. Samples that were treated with the bioadsorbent and initially contained sub-saturating concentrations of LMW heparin displayed the clotting kinetics of blood not treated with heparin. The data confirm that agarose-immobilized RAGE can neutralize the anticoagulant activity of LMW heparin.

### Example 7

### Neutralization of the Anticoagulant Activity of LMW Heparin Using Soluble RAGE

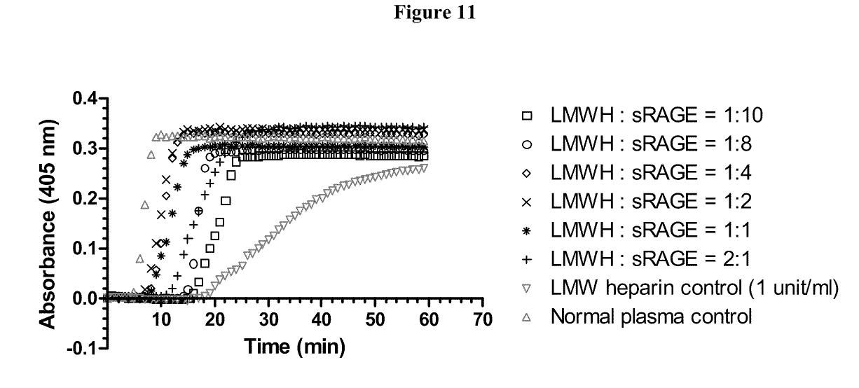

The re-calcified plasma clotting assay was used to demonstrate the ability of soluble RAGE (sRAGE) to neutralize the anticoagulant activity of LMW heparin. The plasma samples were spiked with 1 unit/ml of LMW heparin and varying concentrations of sRAGE so that the molar ratios of LMW heparin to sRAGE were 1:10, 1:8, 1:4, 1:2, 1:1 and 2:1. The samples were incubated at 37° C. for one hour and then tested with the clotting assay. The data are shown in FIG. 11. When the molar ratios of LMW heparin to sRAGE are 1:4, 1:2 and 1:1, the clotting kinetics are very similar to the normal plasma control. The data confirm that sRAGE is able to neutralize the anticoagulant activity of LMW heparin and that it can neutralize equal molar amounts of LMW heparin.

### Example 8

### Heparin Neutralization with Soluble Rage Measured Via Whole Blood Re-Calcification Time

To determine the amount of heparin neutralization, whole blood re-calcification times (WBRT) were measured by using a Hemochron™ 801 clot timer machine. In this assay, 200 μl of citrated blood were spiked with 1 unit/ml heparin and varying concentration of soluble RAGE with molar ratios of heparin to RAGE set as 1:10, 1:8, 1:6, 1:4, 1:2, 1:1 and 2:1. The samples were then incubated at 37° C. for one hour. After that, the blood samples were added to Hemochron™ ACT test tubes containing glass particles and 200 μl of 25 mM CaCl₂ was also added. After starting the Hemochron™ 801 clot timer machine, the test tubes were gently mixed for 10 seconds by flicking the bottom of the tube 5-7 times. The test tubes were then inserted into the test well of the Hemochron™ 801. The time required for a clot to form was recorded and the percentage of unneutralized heparin was calculated by this equation:

$$unneutralized heparin = \frac{( WBRT - {WBRT}_{baseline ( plasma control )} )}{( \begin{matrix}{WBRT}_{maximum ( heparin control )} - \\ {WBRT}_{baseline ( plasma control )}\end{matrix} )}$$

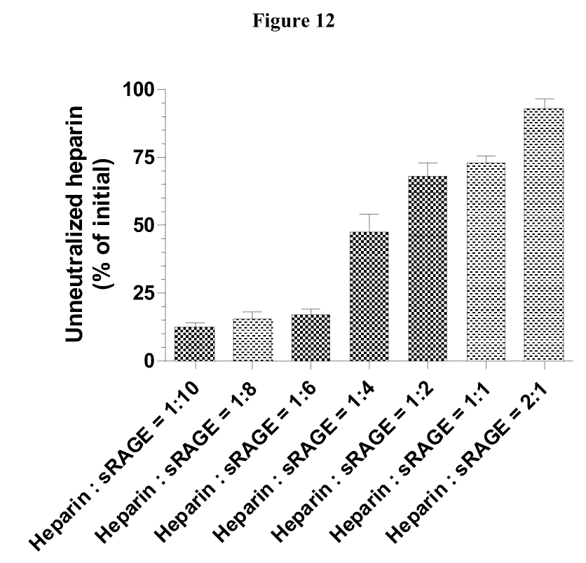

FIG. 12 shows that a 1:6 molar ratio of heparin to soluble RAGE is sufficient to neutralize a significant amount of heparin's anticoagulant activity, which is consistent with the result from the plasma re-calcification clotting assay.

It should be understood that the foregoing disclosure emphasizes certain specific embodiments of the invention and that all modifications or alternatives equivalent thereto are within the spirit and scope of the invention as set forth in the appended claims.

### Example 9

### LMW Heparin Neutralization with Soluble RAGE Measured Via Anti-Xa Assay

To monitor the anticoagulant activity of LMW heparin, anti-Xa assay was more reliable than whole blood re-calcification time assay. In this assay, platelet poor plasma were spiked with 1 unit/ml LMW heparin (Lovenox®) and varying concentration of soluble RAGE with molar ratios of LMW heparin to RAGE set as 1:10, 1:8, 1:6, 1:4, 1:2, 1:1 and 2:1. The samples were incubated at 37° C. for one hour. The concentrations of active LMW heparin remaining in the plasma samples were measured by Actichrome® heparin (anti-fXa) assay (American Diagnostica Inc., Stamford, Conn.) according to the product manual.

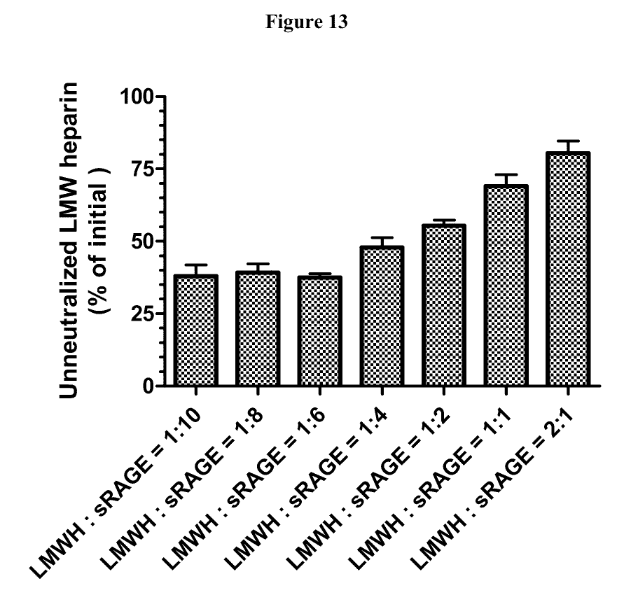

FIG. 13 shows that a 1:6 molar ratio of LMW heparin to soluble RAGE is able to neutralize 64% of LMW heparin's anticoagulant activity, which is less efficient than the neutralization of heparin by soluble RAGE.

### Example 10

### Removal of Heparin with Hollow Fiber Device

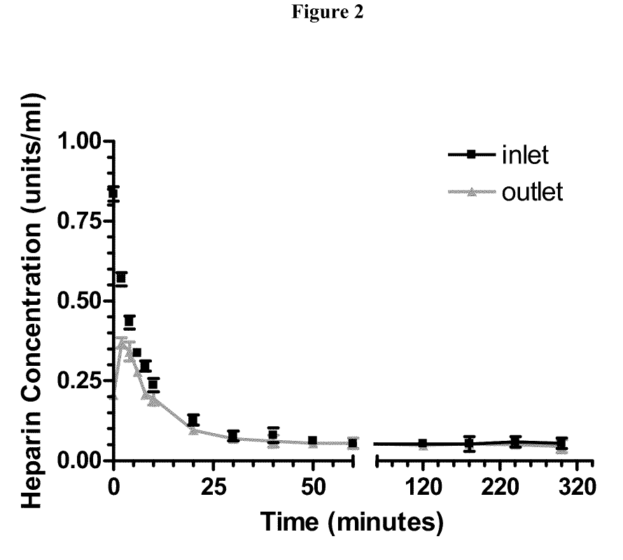

A hollow fiber device was used for testing the ability of immobilized RAGE to remove heparin at large scale. The device has a selective-permeable hollow fiber bundle (˜4000 fibers) inside the shell compartment. The diameter of the device is 1 and ⅜ inch and the length is 9 and ½ inch. The volume of the inner fiber compartment is ˜30 ml and the volume of the outer shell compartment is ˜110 ml. 30 ml of agarose-immobilized RAGE were confined in the outer compartment of the device and 500 ml of heparin solution (1 unit/ml) were circulated through the fibers at a flow rate of 250 ml/min. Samples were taken out at both the inlet and outlet of the fibers at varying time points and heparin concentrations of the samples were measured with Azure II dye. The kinetics of heparin removal are plotted in FIG. 2. The kinetics from the inlet follows a two phase exponential decay. All heparin is cleared out of the solution within one hour of circulation.

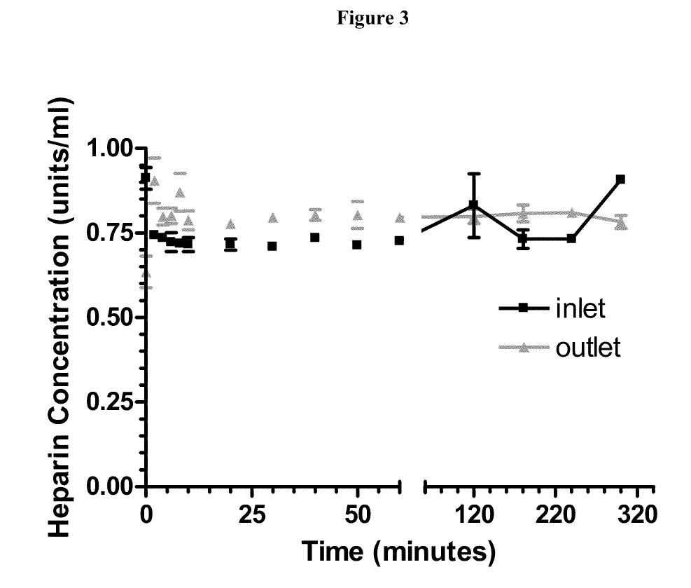

To evaluate the specificity of agarose-immobilized RAGE for heparin binding, the device was packed with 30 ml of plain agarose beads without any RAGE. Similarly, 500 ml of heparin solution (1 unit/ml) was run through the device at flow rate of 250 ml/min. As shown in FIG. 3, there is only 20% reduction in heparin concentration during the first few minutes of run, which may be contributed by the system dilution. The data confirm that complete clearance of heparin from the device is due to the immobilized RAGE.

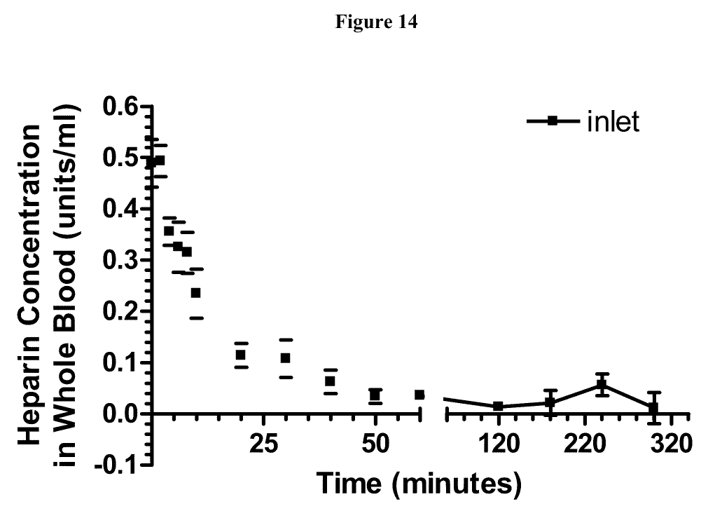

To mimic clinical application, the removal of heparin from human blood with the device was tested. 450 ml of human blood with ACD was spiked with 0.5 unit/ml heparin and circulated through the device. At different time points, blood samples were taken out from the inlet of the device. Concentrations of heparin remained in the blood samples were measured by whole blood re-calcification time assay time assay. The data are shown in FIG. 14. The kinetics follows a two phase exponential decay and all heparin is cleared out of the blood samples within one hour of circulation.

### Example 11

### Removal of LMW Heparin with Hollow Fiber Device

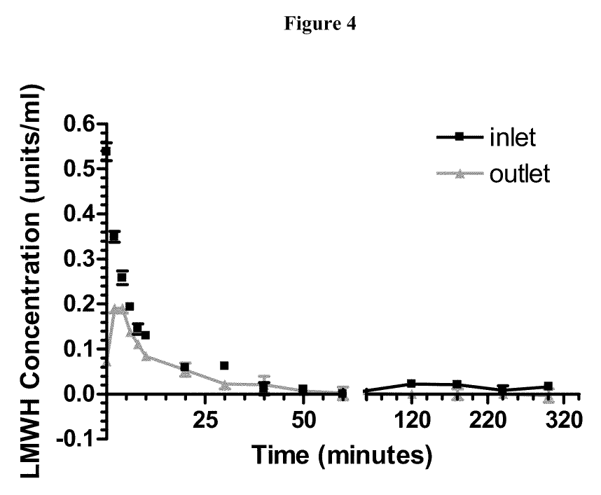

The same hollow fiber device as described in Example 10 was used to test the ability of immobilized RAGE to remove LMW heparin. The beads were stripped with 2 M NaCl for half an hour followed by intense washing with PBS buffer. After that, 500 ml of LMW heparin solution (0.5 unit/ml Lovenox®) was run through the device at flow rate of 250 ml/min. Samples were taken out at both the inlet and outlet of the fibers at varying time points and the LMW heparin concentrations were measured with Azure II dye. The kinetics of LMW heparin removal are plotted in FIG. 4. The kinetics from the inlet follows a two phase exponential decay. All LMW heparin is cleared out of the solution within one hour of circulation. The data also confirm that the same device can be regenerated and the capability to remove heparin is remained after regeneration.

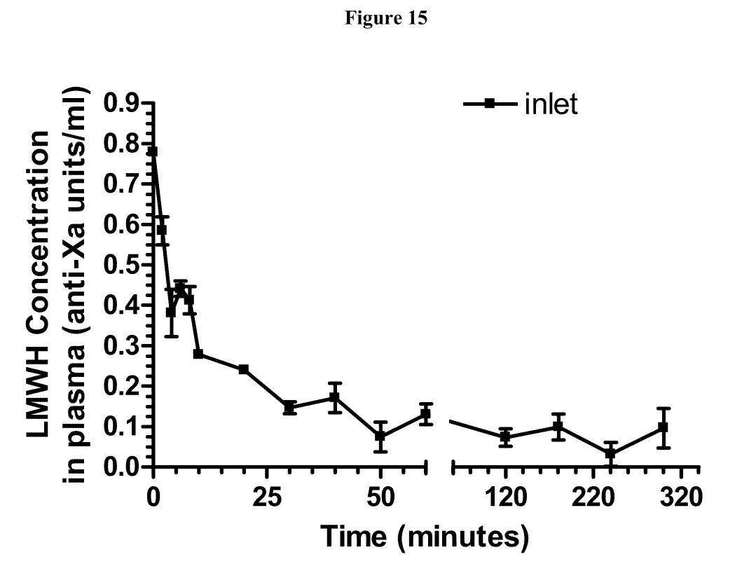

To test the capability of the device for removing LMW heparin from human blood, 450 ml of human blood with ACD was spiked with 0.5 unit/ml LMW heparin (Lovenox®) and circulated through the device. At different time points, blood samples were taken out from the inlet of the device and plasma were isolated. Concentrations of LMW heparin remained in the plasma samples were measured by anti-Xa assay. The data are shown in FIG. 15. The kinetics data can be fit to a two-phase exponential decay and all LMW heparin is cleared out of the blood samples within one hour of circulation.

1. A method for removing heparin from a fluid sample taken from a patient in need of neutralization of heparin anticoagulant activity, the method comprising the steps of:
(a) extracorporeally contacting the fluid sample with a Receptor for Advanced Glycation Endproduct (RAGE) or portion thereof under conditions sufficient to bind heparin, thereby creating a substantially heparin depleted fluid sample, and
(b) returning the heparin depleted fluid sample into the patient.

2. A method for removing heparin from a fluid sample comprising heparin, the method comprising the step of contacting the fluid sample with a Receptor for Advanced Glycation Endproduct (RAGE) or portion thereof under conditions sufficient to bind heparin, thereby creating a substantially heparin depleted fluid sample.

3. A method for neutralizing the anticoagulant activity of heparin in a patient, comprising administering a pharmaceutical composition comprising a Receptor for Advanced Glycation Endproduct (RAGE), or portion thereof and a suitable carrier to a patient in an amount sufficient to substantially bind heparin in the patient, thereby substantially removing heparin from the patient.

4. The method according to claim 1, wherein the heparin comprises low molecular weight heparin (LMW heparin).

5. The method according to claim 1, wherein the heparin comprises a natural or synthetic polysaccharide of heparin.

6. The method according to claim 1, wherein the substantial removal refers to 75%, 77.5%, 80%, 82.5%, 85%, 87.5, 90%, 92.5%, 95%, 97.5%, or 100% removal of heparin, LMW heparin, or polysaccharide of heparin.

7. The method according to claim 1, wherein the RAGE comprises soluble RAGE (sRAGE).

8. The method according to claim 7, wherein the sRAGE is immobilized onto a substrate.

9. The method according to claim 1, wherein the RAGE or portion thereof immobilized onto a substrate.

10. The method according to claim 1 wherein the heparin has a molecular weight ranging from about 5 kDa to about 40 kDa.

11. The method according to claim 1 wherein the LMW heparin has a molecular weight ranging from about 1500 Da to about 9000 Da.

12. The method according to claim 1 wherein the RAGE is conjugated to a water soluble macromolecule.

13. The method according to claim 7 wherein the sRAGE is conjugated to a water soluble macromolecule.

14. The method according to claim 9, wherein the substrate is selected from the group consisting of a particle, a membrane, and a polymeric compound.

15. The method according to claim 14, wherein the polymeric compound is agarose.

16. The method according to claim 1 wherein the RAGE is selected from the group consisting of SEQ ID NO: 4, SEQ ID NO: 5, SEQ ID NO: 6, SEQ ID NO: 8, SEQ ID NO: 9 and SEQ ID NO:13.

17. The method according to claim 1 wherein the RAGE is encoded by a polynucleotide, the polynucleotide selected from the group consisting of SEQ ID NO: 1, SEQ ID NO: 2, SEQ ID NO: 3, SEQ ID NO: 7, SEQ ID NO:12 and complements thereof.
